(19) United States
(12) Patent Application Publication
(10) Pub. No.: US 2007/0255286 A1
(43) Pub. Date: Nov. 1, 2007

(54) Devices, apparatus, and methods for improved disc augmentation

(75) Inventor: Hai H. Trieu, Cordova, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/412,558

(22) Filed: Apr. 27, 2006

(51) Int. Cl. A61B 17/58; A61B 17/60

(52) U.S. Cl. 606/90; 623/17.11

(57) ABSTRACT

A system for controlling a nucleus pulposus augmentation procedure for an intervertebral disc comprises a powered actuation device and a control device for controlling an operating parameter of the actuation device. The system further comprises a space creating instrument including a spacing portion for forming a space within the nucleus pulposus of the intervertebral disc and a delivery instrument for delivering a material to the space. The space creating instrument is activated by the powered actuation device to expand the spacing portion with the material to create the space within the nucleus pulposus of the intervertebral disc.

### BACKGROUND

Within the spine, the intervertebral disc functions to stabilize and distribute forces between vertebral bodies. The intervertebral disc comprises a nucleus pulposus which is surrounded and confined by the annulus fibrosis. Intervertebral discs are prone to injury and degeneration. For example, herniated discs typically occur when normal wear, or exceptional strain, causes a disc to rupture. Degenerative disc disease typically results from the normal aging process, in which the tissue gradually loses its natural water and elasticity, causing the degenerated disc to shrink and possibly rupture.

Intervertebral disc injuries and degeneration are frequently treated by replacing or augmenting the existing disc material. Current methods and instrumentation used for treating the disc require a relatively large hole to be cut in the disc annulus to allow introduction of the implant. After the implantation, the large hole in the annulus must be plugged, sewn closed, or other wise blocked to avoid allowing the implant to be expelled from the disc. Besides weakening the annular tissue, creation of the large opening and the subsequent repair adds surgical time and cost. A need exists for devices, instrumentation, and methods for implanting an intervertebral implant using minimally invasive surgical techniques. A need also exists for a system and methods to control minimally invasive surgical instrumentation.

### SUMMARY

In one embodiment, a system for controlling a nucleus pulposus augmentation procedure for an intervertebral disc comprises a powered actuation device and a control device for controlling an operating parameter of the actuation device. The system further comprises a space creating instrument including a spacing portion for forming a space within the nucleus pulposus of the intervertebral disc and a delivery instrument for delivering a material to the space. The space creating instrument is activated by the powered actuation device to expand the spacing portion with the material to create the space within the nucleus pulposus of the intervertebral disc.

In another embodiment, a method for augmenting a nucleus pulposus of an intervertebral disc comprises introducing a spacing device through an opening in an annulus fibrosis of the intervertebral disc, connecting the spacing device to a material delivery instrument, and connecting the material delivery instrument to an actuator. The method further comprises activating the actuator to dispense a material from the material delivery device into the spacing device and controlling the actuator with a control device in accordance with a preprogrammed profile.

In another embodiment, a method for augmenting a nucleus pulposus of an intervertebral disc comprises forming a first opening in an annulus of the intervertebral disc, forming a second opening in the annulus of the intervertebral disc and providing a space creation instrument including an expandable spacing device. The method further comprises introducing the spacing device through the first opening and into the nucleus pulposus and introducing a material delivery instrument through the second opening and into the nucleus pulposus. The method further comprises expanding the spacing device to create a space within the nucleus pulposus, actuating the material delivery instrument to inject a biocompatible material into the space within the nucleus pulposus, and controlling the injection of the biocompatible material with a first preprogrammed profile.

Additional embodiments are included in the attached drawings and the description provided below.

### BRIEF DESCRIPTION OF THE DRAWINGS

FIG. 1 provides a block diagram of an instrument control system employing one embodiment of the present invention.

FIG. 2 provides a block diagram of an input menu used in the instrument control system of FIG. 1.

FIG. 3 provides a linear expansion profile used in the instrument control system of FIG. 1.

FIGS. 4-6 provide curved expansion profile used in the instrument control system of FIG. 1.

FIGS. 7-9 provide a linear expansion profile used in the instrument control system of FIG. 1.

FIG. 10a provides a sine wave expansion profile used in the instrument control system of FIG. 1.

FIG. 10b provides a square wave expansion profile used in the instrument control system of FIG. 1.

FIG. 11 provides a flowchart of a control routine.

FIG. 12 is a sagittal view of a section of a vertebral column.

FIGS. 13-16 are a sequence of views of an intervertebral disc treatment including accessing the nucleus, inserting an expandable device, expanding the expandable device to create a space, and filling the space.

FIGS. 17-18 are sequence views of an intervertebral disc treatment according to another embodiment of the present disclosure.

FIGS. 19-20 are sequence views of an intervertebral disc treatment according to another embodiment of the present disclosure.

FIGS. 21-22 are sequence views of an intervertebral disc treatment according to another embodiment of the present disclosure.

FIGS. 23-24 are sequence views of an intervertebral disc treatment according to another embodiment of the present disclosure.

FIGS. 25-26 are sequence views of an intervertebral disc treatment according to another embodiment of the present disclosure.

FIG. 27 provides a view of an intervertebral disc treatment according to another embodiment of the present disclosure.

### DETAILED DESCRIPTION

The present disclosure relates generally to devices, methods and apparatus for augmenting an intervertebral disc, and more particularly, to systems for controlling instrumentation for minimally invasive access procedures. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

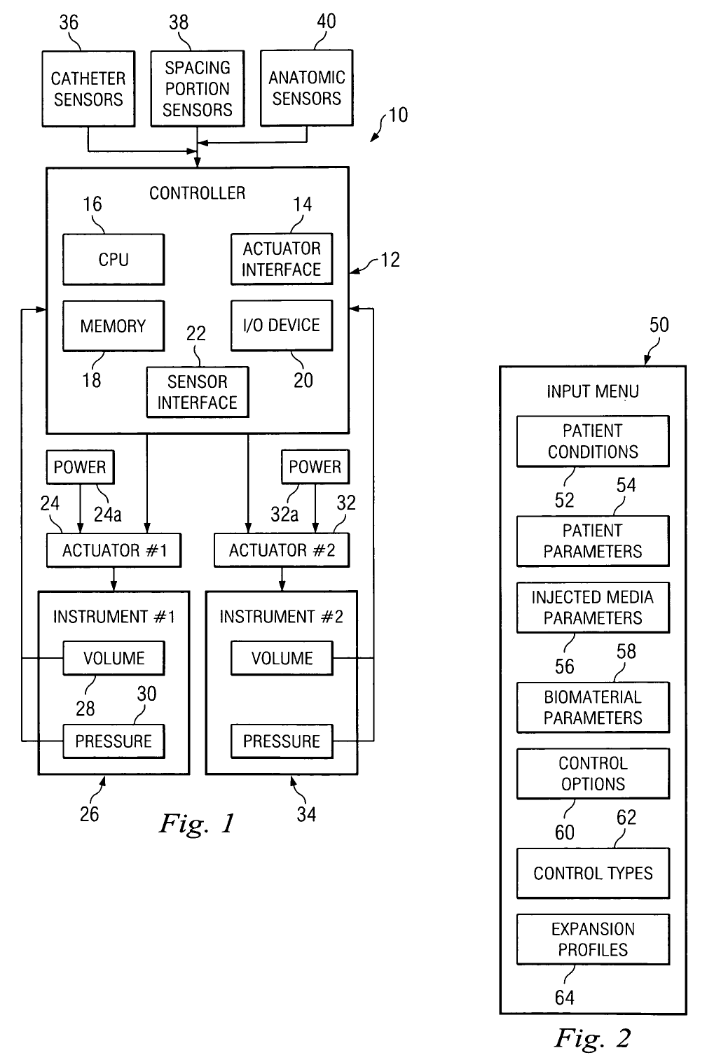

Referring first to FIG. 1, the reference numeral 10 designates an instrument control system including a controller 12 for controlling intervertebral disc augmentation instrumentation and for processing treatment parameter input data and sensor feedback data. The controller 12 may, for example, include an actuator interface component 14, a central processing unit (“CPU”) 16, a memory component 18, and an input/output device 20 such as a monitor or a keyboard. The controller 12 may further include a sensor interface component 22.

The controller 12 may be connected to an actuator 24 such as a motor which may be connected to a disc augmentation instrument 26. It is understood that the motor 24 may be connected to or integral with the instrument 26. The instrument 26 may include various sensors such as a volume sensor 28 and a pressure sensor 30. The sensors 28, 30 may be in communication with the controller 12 by, for example, a direct connection, a biotelemetry connection, or a private or public network connection. A second motor 32 may be connected to an instrument 34 which may have similar sensors to instrument 26. Additional sensors may be located remotely from the instruments 26, 34 including conduit sensors 36, spacing portion sensors 38, and anatomic sensors 40. The actuators 24, 32 may be powered by power supplies 24a, 32a, respectively. The power supplies may be powered by battery power, direct electrical power, pneumatic power, etc.

Referring now to FIG. 2, in this embodiment an input menu 50 may be used to determine the output of the controller 12. The input menu 50 may allow a user, such as a physician, to input data pertinent to an intervertebral disc augmentation surgery. The menu 50 may allow the input of criteria such as patient conditions 52 including data related to intervertebral disc surgery such as nature of pathology, symptoms, disc height, disc volume, disc hydration level, previous surgeries, and pain tolerance level. The menu 50 may further allow the input of patient parameters 54 such as patient height, weight, and age. The menu 50 may further allow the input of injected media parameters 56 such as type of media. The menu 50 may further allow the input of biomaterial parameters 58 such as type of biomaterial, viscosity, and whether the biomaterial or spacing portion will remain within the disc as an implant. The menu 50 may further allow the input of control options 60 such selecting between manual or automatic control. The menu 50 may further allow the input of control types 62 such as pressure, volume, time, and rate. The menu 50 may further allow the input of expansion profiles 64.

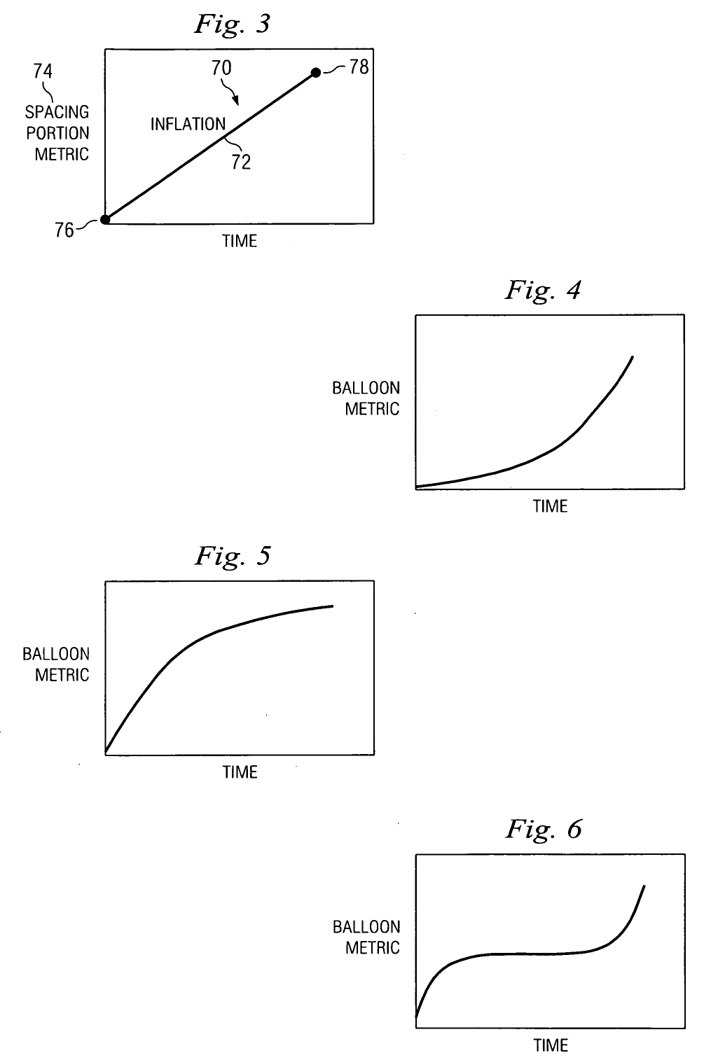

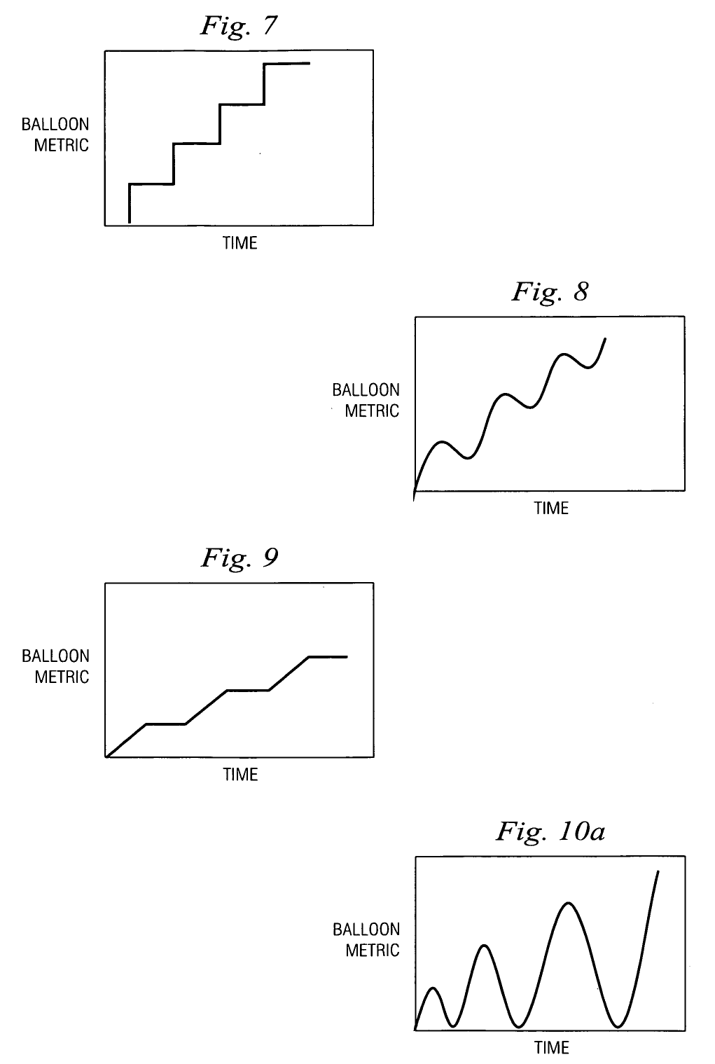

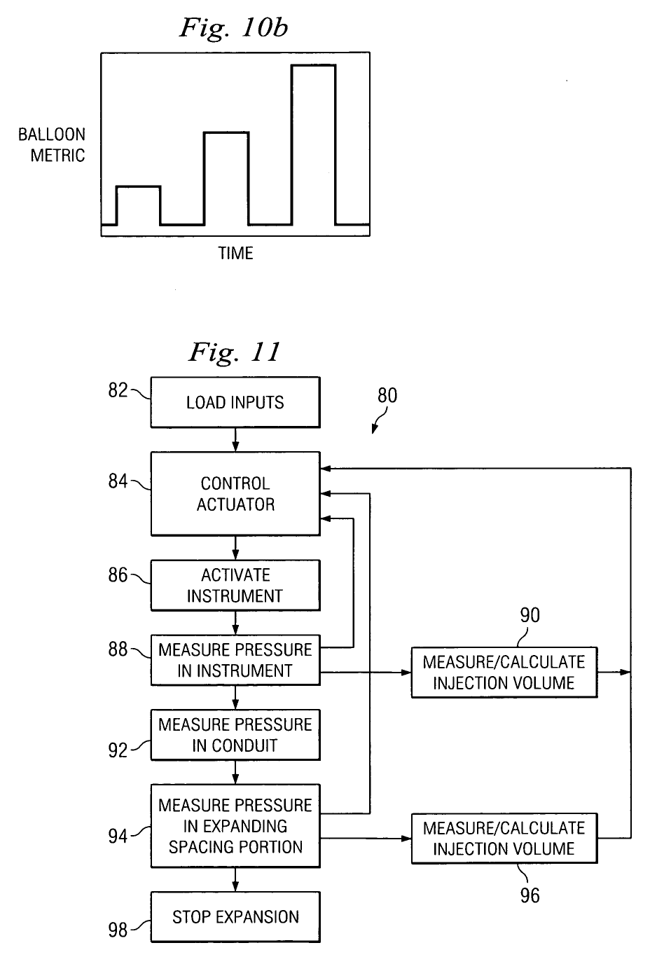

The expansion profiles 64 may be used to control the pressure in a disc spacing portion or volume of material dispensed to the spacing portion. Referring now to FIG. 3, one exemplary expansion profile 70 may have a linear relationship 72 between a spacing portion metric 74, such as pressure, and the elapsed time for an expansion procedure. At a beginning time 76, the spacing portion may be unexpanded, and at an end time 78, the spacing portion may be expanded to an optimum level for a given patient. As shown in FIGS. 4-6, in other embodiments, expansion profiles may be curved. As shown in FIGS. 7-9, in other embodiments, expansion profiles may be any of a variety of step-up functions. As shown in FIG. 10a, in another embodiment, an expansion profile may be a type of sine wave. As shown in FIG. 10b, in another embodiment, an expansion profile may be a type of square wave.

Referring now to FIG. 11, a process 80 for implementing the system 30 of FIG. 1 may begin with the step 82 of accessing the input menu 50 and entering the data regarding patient condition 52, patient parameters 54, injected media parameters 56, biomaterial parameters 58, control options 60, control types 62, and/or expansion profiles 64. Additional or alternative data may also be entered regarding the patient or surgical procedure. It is understood that the data for the input menu 50 may be changed or provided as needed throughout the surgical procedure. The expansion profiles, such as those shown in FIGS. 3-10b, may be preprogrammed into the controller 12 or may be uniquely created and entered by a user of the system 10.

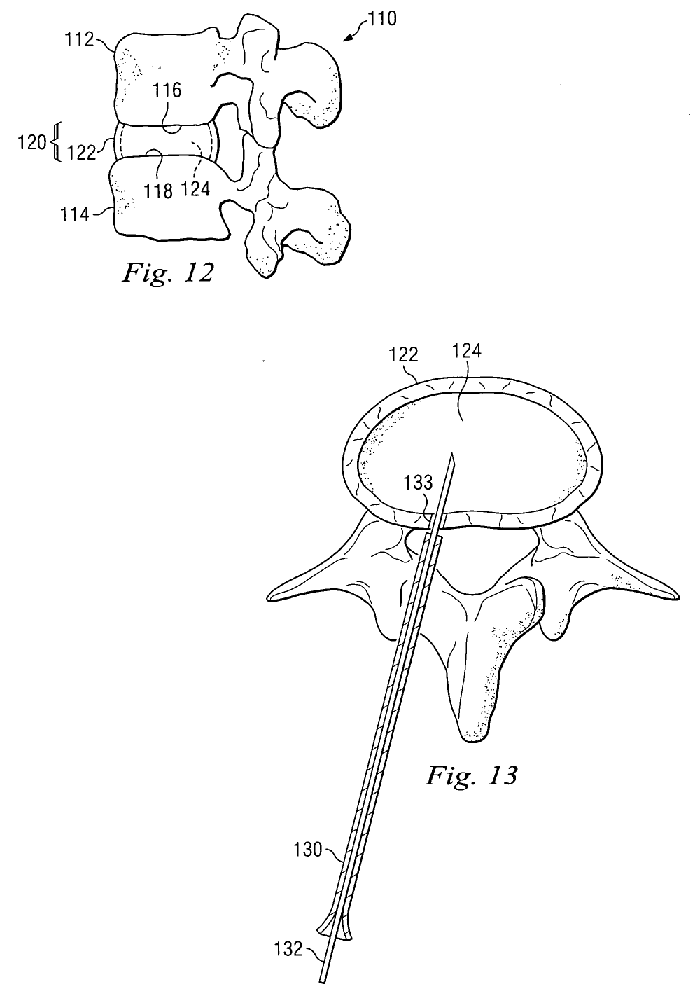

Referring now to FIG. 12, the system 10 may be used to control instrumentation used to augment a vertebral joint section 110 of a vertebral column. Methods and instrumentation for augmenting a vertebral joint are described in further detail in U.S. patent application Ser. No. ______, entitled “DEVICES, APPARATUS, AND METHODS FOR BILATERAL APPROACH TO DISC AUGMENTATION” (Attorney Docket No. 31132.513), filed concurrently herewith and incorporated by reference herein.

The joint section 110 includes adjacent vertebral bodies 112, 114. The vertebral bodies 112, 114 include endplates 116, 118, respectively. An intervertebral disc space 120 is located between the endplates 116, 118, and an annulus 122 surrounds the space 120. In a healthy joint, the space 120 contains a nucleus pulposus 124.

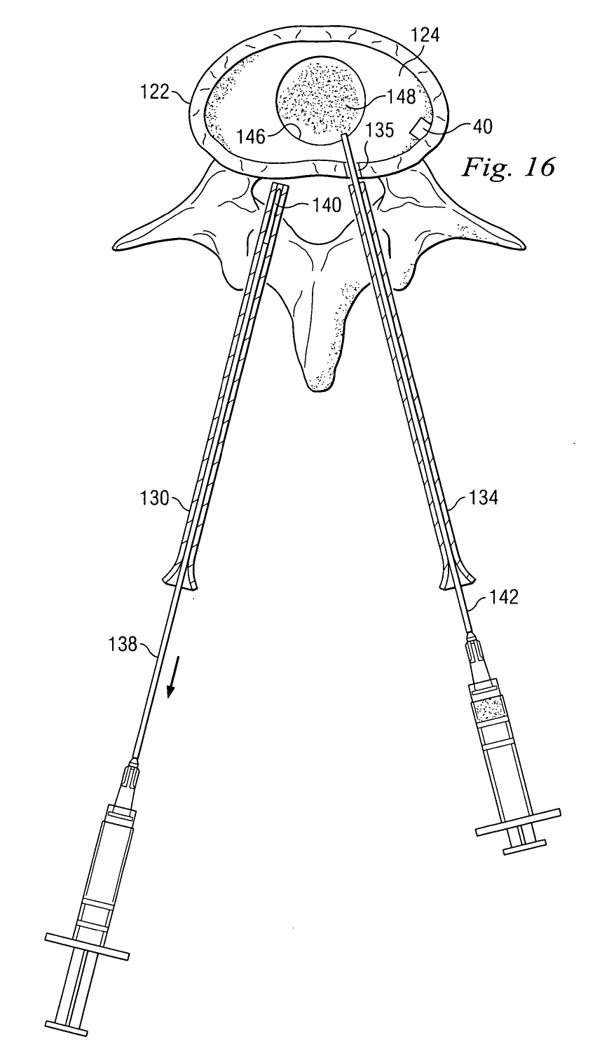

Referring now to FIGS. 13-16, in this bilateral approach, the nucleus 124 may be accessed by inserting a cannula 130 into the patient and locating the cannula at or near the annulus 122. An accessing instrument 132, such as a trocar needle or a K-wire is inserted through the cannula 130 and used to penetrate the annulus 122, creating an annular opening 133. This accessing procedure may be repeated at another position on the annulus 122 using a cannula 134 to create an annular opening 135. With the openings 133, 135 created, the accessing instrument 132 may be removed and the cannulae 130, 134 left in place to provide passageway for additional instruments.

In this embodiment, the nucleus is accessed using a posterior bilateral approach. In alternative embodiments, the annulus may be accessed with a lateral approach, an anterior approach, a trans-pedicular/vertebral endplate approach or any other suitable nucleus accessing approach. Although a bilateral approach is described, a unilateral or multi-lateral approach may be suitable. In another alternative embodiment, the nucleus 124 may be accessed through one the of vertebral bodies 112, 114 and through its respective endplate 116, 118. Thus, a suitable bilateral approach to nucleus augmentation may involve a combination approach including an annulus access opening and an endplate access opening.

It is understood that any cannulated instrument including a guide needle or a trocar sleeve may be used to guide the accessing instrument.

In this embodiment, the natural nucleus, or what remains of it after natural disease or degeneration, may remain intact with no tissue removed. In alternative embodiments, partial or complete nucleotomy procedures may be performed.

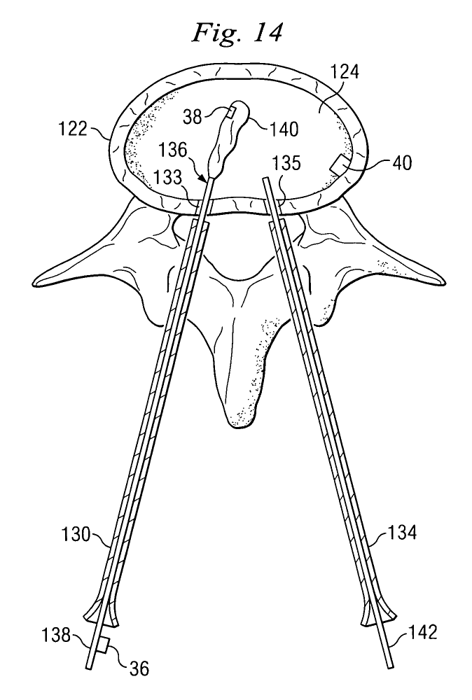

As shown in FIG. 14, in this embodiment, a space creating device 136 having a catheter portion 138 and a spacing portion 140 may be inserted through the cannula 130 and the annular opening 133 into the nucleus 124. A delivery instrument 141, which may be the instrument 26, is connected to the catheter portion 138. In this embodiment, the spacing portion 140 is an expandable device such as a balloon which may be formed of elastic or non-elastic materials. One or more of the sensors 36 may be located in or on the catheter portion 138. One or more of the sensors 38 may be located in or on the spacing portion 140. One or more of the sensors 40 may be located in the disc space 20.

The pattern, size, or shape of the spacing portion 140 can be varied between patients depending on disc condition. The balloon can be of various shapes including conical, spherical, square, long conical, long spherical, long square, tapered, stepped, dog bone, offset, or combinations thereof. Balloons can be made of various polymeric materials such as polyethylene terephthalates, polyolefins, polyurethanes, nylon, polyvinyl chloride, silicone, polyetheretherketone, polylactide, polyglycolide, poly(lactide-co-glycoli-de), poly(dioxanone), poly(.epsilon.-caprolactone), poly(hydroxylbutyrate), poly(hydroxylvalerate), tyrosine-based polycarbonate, polypropylene fumarate or combinations thereof. Additionally, the expandable device may be molded or woven.

In an alternative embodiment, the spacing portion may be mechanical instrument such as a probe or a tamp. A mechanically actuated deformable or expandable instrument which may deform via hinges, springs, shape memory material, etc. may also be used as a spacing portion. In some embodiments, the passage of the spacing portion may be aided with a more rigid guide needle or cannula which will accompany the spacing portion through the cannula and the annulus opening. This guide may be removed after the spacing portion is located within the nucleus 124.

As also shown in FIG. 14, a delivery instrument 142 may be passed through the cannula 134, through the annular opening 135, and into the nucleus 124. The delivery instrument 142 may be an injection needle or other material delivery instrument and may be blunt to avoid puncture or damage to the spacing portion 140.

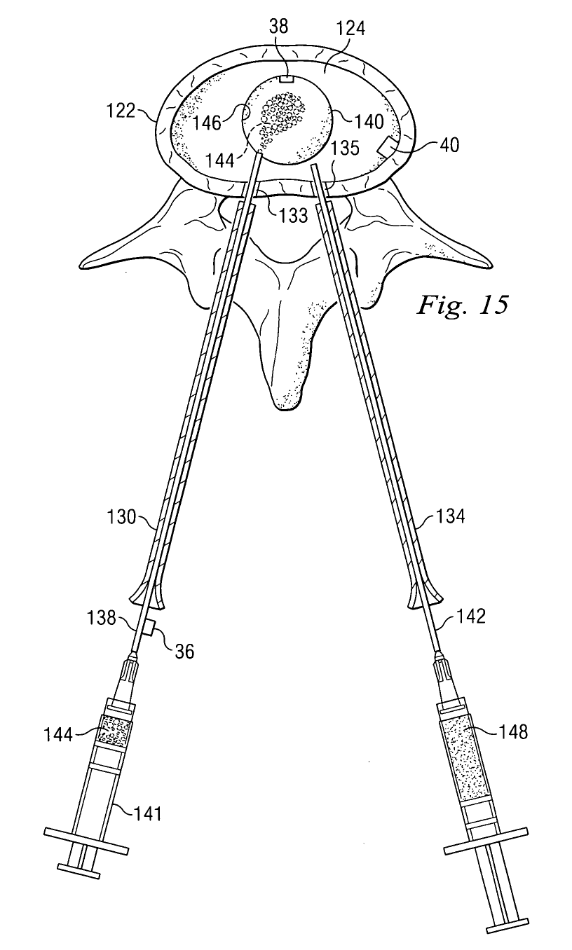

Referring now to FIGS. 15, an inflation medium 144 may be pressurized and injected or otherwise passed through the catheter portion 138 of the space creating device 136 to pressurize and inflate the spacing portion 140. The inflation medium 144 may be a saline and/or radiographic contrast medium such as sodium diatrizoate solution sold under the trademark Hypaque® by Amersham Health, a division of GE Healthcare (Amersham, UK). The inflation medium 144 may be injected under pressure supplied by a hand, electric, or other type of powered pressurization device.

The injection of the inflation medium 144 may be controlled using a control process 80. Referring again to FIG. 11, in step 84 of the process 80, the controller 12 of the system 10 may be used to control the motor 24. The motor 24 may actuate the instrument 26 which in this embodiment is the injector 141. Based on the input data 52-62 and the selected expansion profile 64, the injection 141 is powered to dispense the inflation medium 144 according to the selected profile. At steps 88 and 90, as the inflation medium 144 is dispensed, the pressure sensor 30 and the volume sensor 28 may provide feedback data to the controller 12, allowing the controller to adjust the motor speed to maintain the selected profile. As shown in step 90, calculations may be performed to determine the volume of the material 144 dispensed. At step 92, the conduit sensors 36 may provide feedback data to the controller 12 regarding the pressure in catheter portion 138. At step 94, the spacing portion sensors 38 may provide feedback data to the controller regarding the pressure or material volume in spacing portion 140. As shown in step 96, calculations may be performed to determine the volume of the material 144 dispensed to the spacing portion. At step 98, the controller 12 stops the actuator 24 from dispensing the inflation medium 144 according to the expansion profile selected. The inflation medium 144 may later be removed to deflate the spacing portion 140. Although only expansion profiles have been described, the controller 12 may also activate the actuator 24 to deflate the spacing portion according to a predetermined profile.

As the spacing portion 140 is inflated according to the selected expansion profile, a space 46 is created in the nucleus tissue with the surrounding nucleus tissue becoming displaced or stretched. The inflation may also cause the intradiscal pressure to increase. Both the pressure increase and the direct expansion of the portion 140 may cause the endplates 116, 118 to distract. A pressure gauge and/or a pressure limiter may be used to avoid over inflation or excessive injection.

In an alternative embodiment, the space creating portion may be disposed within the annular opening 133 such that as the space creating portion is expanded, the opening becomes stretched or dilated by the space creating device.

After the space 146 is created, the space creating portion 140 is deflated leaving the space 146 to be filled by a biocompatible material 48 injected from the delivery instrument 142. The injection of the material 148 may be facilitated by using a pressurization device and monitoring gauge. The material 148 may be injected after the space creating portion 140 has been deflated and removed or may be injected while the space creating portion 140 is being deflated and removed. For example, the biomaterial 148 may become increasingly pressurized while the pressure in the space creating portion 140 is lowered. In some procedures, the material 148 may be injected before the space creating portion 140 is removed.

The injection of the material 148 may also be controlled using the controller 12 and a process similar to the process described for FIG. 11. The delivery instrument 142 may be the instrument 34 controlled by the actuator 32. The actuator 32 may inject the material 148 by following a selected expansion profile. The term “expansion profile” is not limited to profiles for expanding balloons and other inflatable devices, but rather, may more broadly apply to any preprogrammed control profile for operating a delivery device, including a profile for dispensing material.

Examples of biocompatible materials 148 which may be used for disc augmentation include natural or synthetic and resorbable or non-resorbable materials. Natural materials include various forms of collagen that are derived from collagen-rich or connective tissues such as an intervertebral disc, fascia, ligament, tendon, skin, or demineralized bone matrix. Material sources include autograft, allograft, xenograft, or human-recombinant origin materials. Natural materials also include various forms of polysaccharides that are derived from animals or vegetation such as hyaluronic acid, chitosan, cellulose, or agar. Other natural materials include other proteins such as fibrin, albumin, silk, elastin and keratin. Synthetic materials include various implantable polymers or hydrogels such as silicone, polyurethane, silicone-polyurethane copolymers, polyolefin, polyester, polyacrylamide, polyacrylic acid, polyvinyl alcohol, polyethylene oxide, polyethylene glycol, polylactide, polyglycolide, poly(lactide-co-glycolide), poly(dioxanone), poly(.epsilon.-caprolactone), poly(hydroxylbutyrate), poly(hydroxylvalerate), tyrosine-based polycarbonate, polypropylene fumarate or combinations thereof. Suitable hydrogels may include poly(vinyl alcohol), poly(acrylic acids), poly(methacrylic acids), copolymers of acrylic acid and methacrylic acid, poly(acrylonitrile-acrylic acid), polyacrylamides, poly(N-vinyl-2-pyrrolidone), polyethylene glycol, polyethyleneoxide, polyacrylates, poly(2-hydroxy ethyl methacrylate), copolymers of acrylates with N-vinyl pyrrolidone, N-vinyl lactams, polyurethanes, polyphosphazenes, poly(oxyethylene)-poly(oxypropylene) block polymers, poly(oxyethylene)-poly(oxypropylene) block polymers of ethylene diamine, poly(vinyl acetate), and sulfonated polymers, polysaccharides, proteins, and combinations thereof.

The selected biocompatible material may be curable or polymerizable in situ. The biocompatible material may transition from a flowable to a non-flowable state shortly after injection. One way to achieve this transition is by adding a crosslinking agent to the biomaterial before, during, or after injection. The biocompatible material in its final state may be load-bearing, partially load-bearing, or simply tissue augmenting with minimal or no load-bearing properties.

Proteoglycans may also be included in the injectable biocompatible material 48 to attract and/or bind water to keep the nucleus 24 hydrated. Regnerating agents may also be incorporated into the biocompatible material. An exemplary regenerating agent includes a growth factor. The growth factor can be generally suited to promote the formation of tissues, especially of the type(s) naturally occurring as components of an intervertebral disc. For example, the growth factor can promote the growth or viability of tissue or cell types occurring in the nucleus pulposus, such as nucleus pulposus cells and chondrocytes, as well as space filling cells, such as fibroblasts and connective tissue cells, such as ligament and tendon cells. Alternatively or in addition, the growth factor can promote the growth or viability of tissue types occurring in the annulus fibrosis, as well as space filling cells, such as fibroblasts and connective tissue cells, such as ligament and tendon cells. An exemplary growth factor can include transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor P protein, bone morphogenetic protein, fibroblast growth factor, platelet-derived growth factor, insulin-like growth factor, or any combination thereof.

Therapeutic or biological agents may also be incorporated into the biomaterial. An exemplary therapeutic or biological agent can include a soluble tumor necrosis factor α-receptor, a pegylated soluble tumor necrosis factor α-receptor, a monoclonal antibody, a polyclonal antibody, an antibody fragment, a COX-2 inhibitor, a metalloprotease inhibitor, a glutamate antagonist, a glial cell derived neurotrophic factor, a B2 receptor antagonist, a substance P receptor (NK1) antagonist, a downstream regulatory element antagonistic modulator (DREAM), iNOS, a inhibitor of tetrodotoxin (TTX)-resistant Na+-channel receptor subtypes PN3 and SNS2, an inhibitor of interleukin, a TNF binding protein, a dominant-negative TNF variant, Nanobodies™, a kinase inhibitor, or any combination thereof.

These regenerating, therapeutic, or biological agents may promote healing, repair, regeneration and/or restoration of the disc, and/or facilitate proper disc function. Additives appropriate for use in the claimed invention are known to persons skilled in the art, and may be selected without undue experimentation.

After the biocompatible material 148 is injected, the delivery instrument 142 may be removed from the cannula 134. If the selected biocompatible material 148 is curable in situ, the instrument 142 may be removed during or after curing to minimize leakage. The openings 133, 135 may be small enough, for example less than 3mm, that they will close or close sufficiently that the injected biocompatible material 148 will remain within the annulus. The use of an annulus closure device such as a suture, a plug, or a material sealant is optional. The cannulae 130, 134 may be removed and the minimally invasive surgical incision closed.

Any of the steps of the method including expansion of the space creating portion 140 and filling the space 146 may be monitored and guided with the aid of imaging methods such as fluoroscopy, x-ray, computed tomography, magnetic resonance imaging, and/or image guided surgical technology such as a Stealth Station™ surgical navigation system (Medtronic, Inc., Minneapolis, Minn.) or a BrainLab system (Heimstetten, Germany).

In an alternative embodiment, the space creating portion may be detachable from the catheter portion and may remain in the nucleus 124 as an implant. In this alternative, the biocompatible material may be injected directly into the space creating portion.

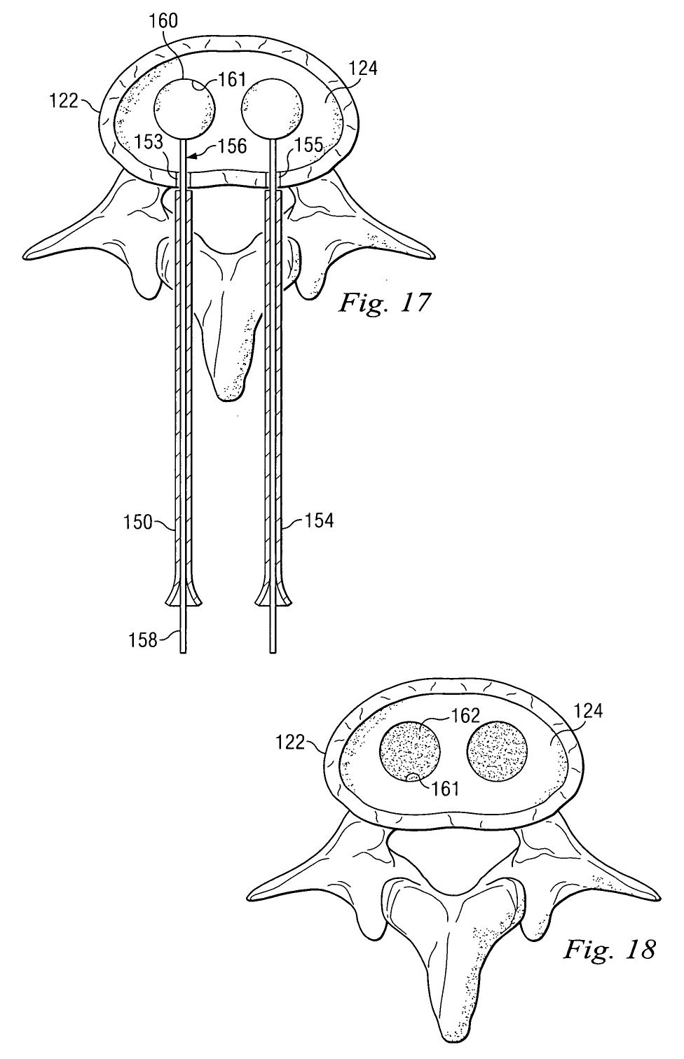

Referring now to FIGS. 17-18, in this embodiment, the nucleus 124 may be accessed by inserting a cannula 150 into the patient and locating the cannula at or near the annulus 122. As described above, an accessing instrument is inserted through the cannula 150 and used to penetrate the annulus 122, creating an annular opening 153. This accessing procedure may be repeated at another position on the annulus 122 using a cannula 154 to create an annular opening 155. With the openings 153, 155 created, the accessing instrument may be removed and the cannulae 150, 154 left in place to provide bilateral passageways for additional instruments. In this embodiment, the natural nucleus, or what remains of it after natural disease or degeneration, may remain intact with no tissue removed. In alternative embodiments, partial or complete nucleotomy procedures may be performed.

As shown in FIG. 17, a space creating device 156 having a catheter portion 158 and a spacing portion 160 may be inserted through the cannula 150 and the annular opening 153 into the nucleus 124. In this embodiment, the spacing portion is an expandable device such as a balloon which may be formed of elastic or non-elastic materials. The characteristics of the balloon may be the same or similar to those described above. The spacing portion may be inflated and removed as described in further detail in U.S. patent application Ser. No. 10/314,396 (“the '396 application”) which is incorporated herein by reference. The space 161 created by the spacing portion may be filled with a biocompatible material 162 using the cannula 154 through the bilateral opening 155 in a manner similar to that described above in FIGS. 13-16 or alternatively, using the same cannula 150 and the opening 153 in a manner similar to that described in the '396 application. The procedure of creating a space in the nucleus 124 may be repeated in another location of the nucleus using the annular opening 155 to pass a space creating device for creating a second space to be filled with a biocompatible material. This procedure may be substantially similar to that described above for creating and filling space 161. The space creation and filling procedures of this embodiment may be controlled with a process and system similar to that described above for FIGS. 1 and 11. Although not shown, sensors similar to those described above for FIGS. 13-16 may be embedded in the instrumentation or disc space to monitor the space creation and filling.

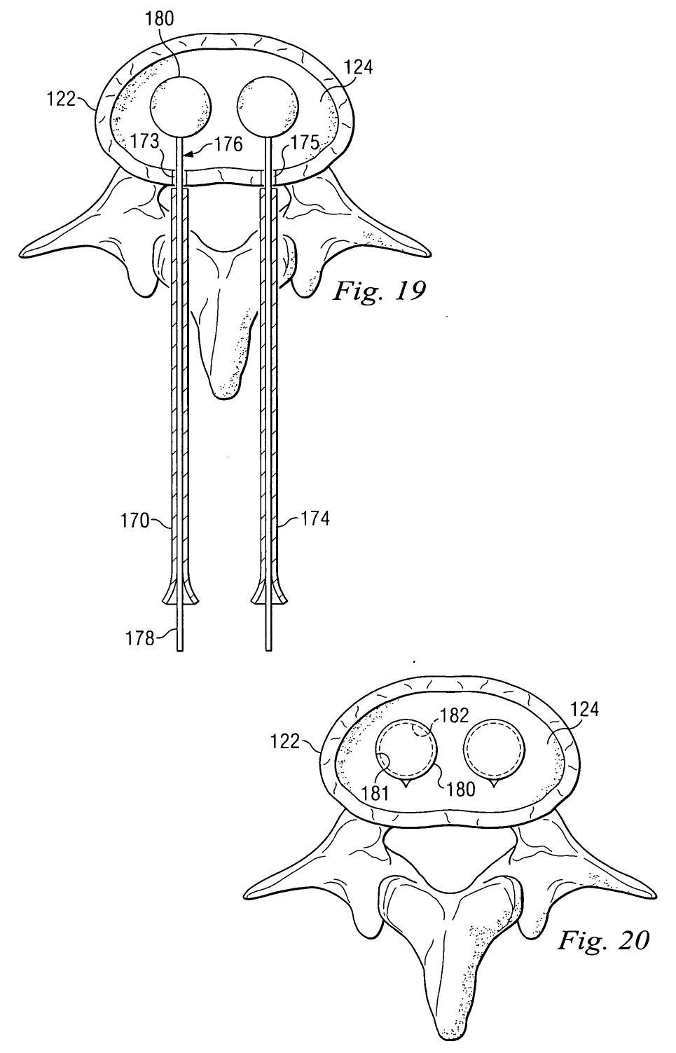

Referring now to FIGS. 19-20, in this embodiment, the nucleus 124 may be accessed by inserting a cannula 170 into the patient and locating the cannula at or near the annulus 122. As described above, an accessing instrument is inserted through the cannula 170 and used to penetrate the annulus 122, creating an annular opening 173. This accessing procedure may be repeated at another position on the annulus 122 using a cannula 174 to create an annular opening 175. With the openings 173, 175 created, the accessing instrument may be removed and the cannulae 170, 174 left in place to provide bilateral passageways for additional instruments. In this embodiment, the natural nucleus, or what remains of it after natural disease or degeneration, may remain intact with no tissue removed. In alternative embodiments, partial or complete nucleotomy procedures may be performed.

As shown in FIG. 19, a space creating device 176 having a catheter portion 178 and a spacing portion 180 may be inserted through the cannula 170 and the annular opening 173 into the nucleus 124. In this embodiment, the spacing portion is an expandable device such as a balloon which may be formed of elastic or non-elastic materials. The characteristics of the balloon may be the same or similar to those described above. The spacing portion 180 may be pressurized and filled with a biocompatible material 182 as described in further detail in the '396 application. In this embodiment, the filled spacing portion 180 may be detached and left within the nucleus pulposus 124 as an implant. The procedure of creating a space in the nucleus 124 may be repeated in another location of the nucleus using the annular opening 155 to pass a spacing portion for creating a second space, filling the spacing portion with a biocompatible material, and detaching the second spacing portion. This procedure may be substantially similar to the procedure for filling the spacing portion 180. In an alternative embodiment, the spacing portion may be filled with a biocompatible material using the cannula 174 and the bilateral opening 175 in a manner similar to that described above for FIGS. 13-16. This delivery of material through the bilateral opening 175 may occur either before or after the spacing portion is detached from the catheter portion of the space creating device. The space creation and filling procedures of this embodiment may be controlled with a process and system similar to that described above for FIGS. 1 and 11. Although not shown, sensors similar to those described above in FIGS. 13-16 may be embedded in the instrumentation or disc space to monitor the space creation and filling.

In other embodiments, spacing portions similar to those described in the previous embodiments may be preformed in various shapes, such as triangular or capsular, to achieve patient-specific goals including compensating for unique nucleus degradation or patient-tailored endplate distraction.

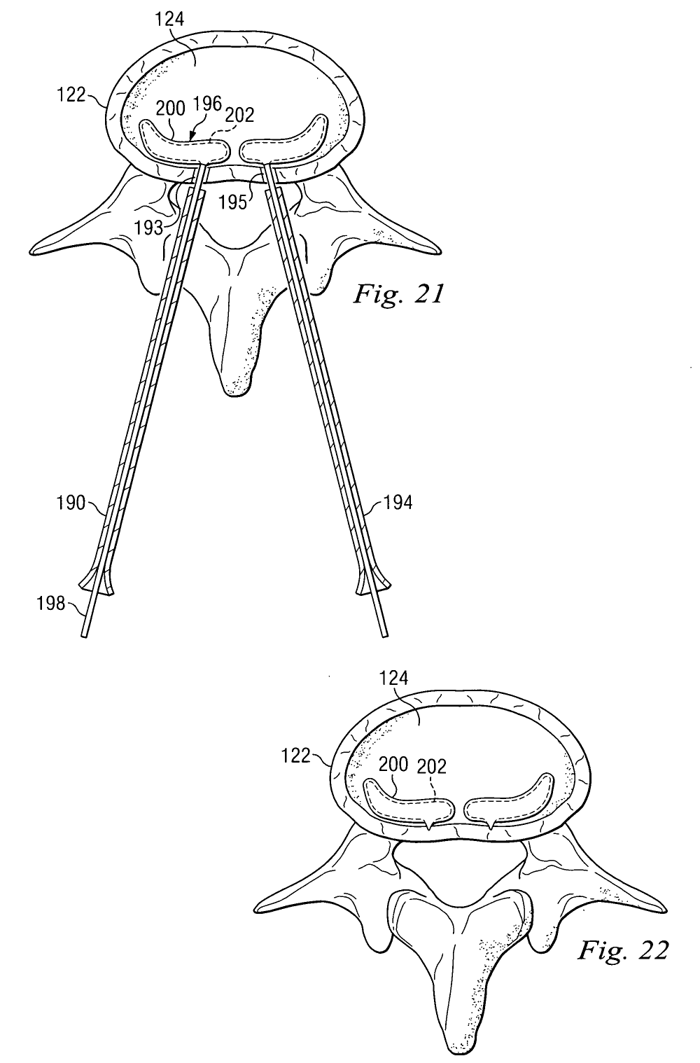

Referring now to FIGS. 21 and 22, in this embodiment, the nucleus 124 may be accessed by inserting a cannula 190 into the patient and locating the cannula at or near the annulus 122. As described above, an accessing instrument is inserted through the cannula 190 and used to penetrate the annulus 122, creating an annular opening 193. This accessing procedure may be repeated at another position on the annulus 122 using a cannula 194 to create an annular opening 195. With the openings 193, 195 created, the accessing instrument may be removed and the cannulae 190, 194 left in place to provide bilateral passageways for additional instruments. In this embodiment, the natural nucleus, or what remains of it after natural disease or degeneration, may remain intact with no tissue removed. In alternative embodiments, partial or complete nucleotomy procedures may be performed.

As shown in FIG. 21, a space creating device 196 having a catheter portion 198 and a spacing portion 200 may be inserted through the cannula 190 and the annular opening 193 into the nucleus 124. In this embodiment, the spacing portion 200 is an expandable device such as a balloon which may be formed of elastic or non-elastic materials. The characteristics of the balloon may be the same or similar to those described above. The balloon may be shaped to fit along the inner contour of the annulus 122. The spacing portion 200 may be pressurized, filled, and detached as described above. The spacing portion 200 may be filled with a biocompatible material 202 using the cannula 194 and the bilateral opening 195 in a manner similar to that described above for FIGS. 13-16 or using the same cannula 190 and the opening 193 in a manner similar to that described in the '396 application. The procedure of creating a space in the nucleus 124 along the annulus 122 may be repeated in another location of the nucleus using the annular opening 155 to pass a space creating device for creating a second implant to be filled with a biocompatible material. This procedure may be substantially similar to that described above for creating and filling spacing portion 200. The implant created by the filled spacing portion 200 and its bilateral counterpart may be contoured to fit along an interior segment of annulus 122. The resulting implant may support a weakened annulus or reinforce a ruptured annulus to reduce or prevent nucleus herniation. The biocompatible material may be selected to optimize support and flexibility. The space creation and filling procedures of this embodiment may be controlled with a process and system similar to that described above for FIGS. 1 and 11. Although not shown, sensors similar to those described above in FIGS. 13-16 may be embedded in the instrumentation or disc space to monitor the space creation and filling.

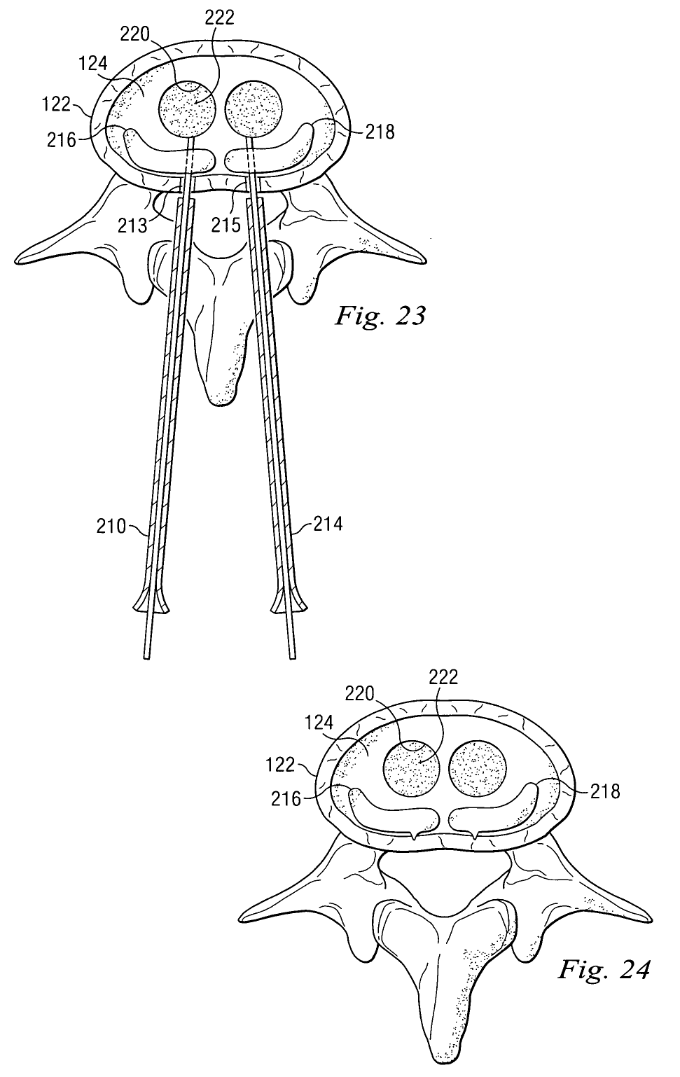

Referring now to FIGS. 23 and 24, in this embodiment, the nucleus 124 may be accessed by inserting a cannula 210 into the patient and locating the cannula at or near the annulus 122. As described above, an accessing instrument is inserted through the cannula 210 and used to penetrate the annulus 122, creating an annular opening 213. This accessing procedure may be repeated at another position on the annulus 122 using a cannula 214 to create an annular opening 215. With the openings 213, 215 created, the accessing instrument may be removed and the cannulae 210, 214 left in place to provide bilateral passageways for additional instruments. In this embodiment, the natural nucleus, or what remains of it after natural disease or degeneration, may remain intact with no tissue removed. In alternative embodiments, partial or complete nucleotomy procedures may be performed.

As shown in FIG. 23, annulus contoured spacing portions 216, 218 may be inserted, detached, and filled as described above in FIG. 21. The resulting implant may support a weakened annulus or reinforce a ruptured annulus to reduce or prevent nucleus herniation. The biocompatible filling material may be selected to optimize support and flexibility. These annulus reinforcing spacing portions 216, 218 may be used in conjunction with the more centralized nucleus spacing procedures described in FIGS. 13-16. In this embodiment, an additional spacing portion may be inserted through the filled spacing portions 216, 218 and expanded within the nucleus 124 to create a space 220. The space 220 may be filled with a biomaterial 222. More spacing portions may be inserted to create additional filled spaces in the nucleus 124. The use of annular spacing portions in conjunction with more centralized spacing portions may help to prevent the more centralized biomaterial and the natural nucleus tissue from migrating through annular defects or openings. The biomaterials selected for filling the various spaces and spacing portions may be the same or different depending upon the desired result. The space creation and filling procedures of this embodiment may be controlled with a process and system similar to that described above for FIGS. 1 and 11. Although not shown, sensors similar to those described above for FIGS. 13-16 may be embedded in the instrumentation or disc space to monitor the space creation and filling.

In an alternative embodiment, a delivery instrument may be inserted through the spacing portions 216, 218 to deposit a biocompatible material directly into the nucleus 124 without creating an additional space within the nucleus. In this embodiment, the spacing portions serve to block migration or expulsion of the biocompatible material through the annulus, however the material may be more dispersed within the nucleus rather than concentrated in a pre-formed space.

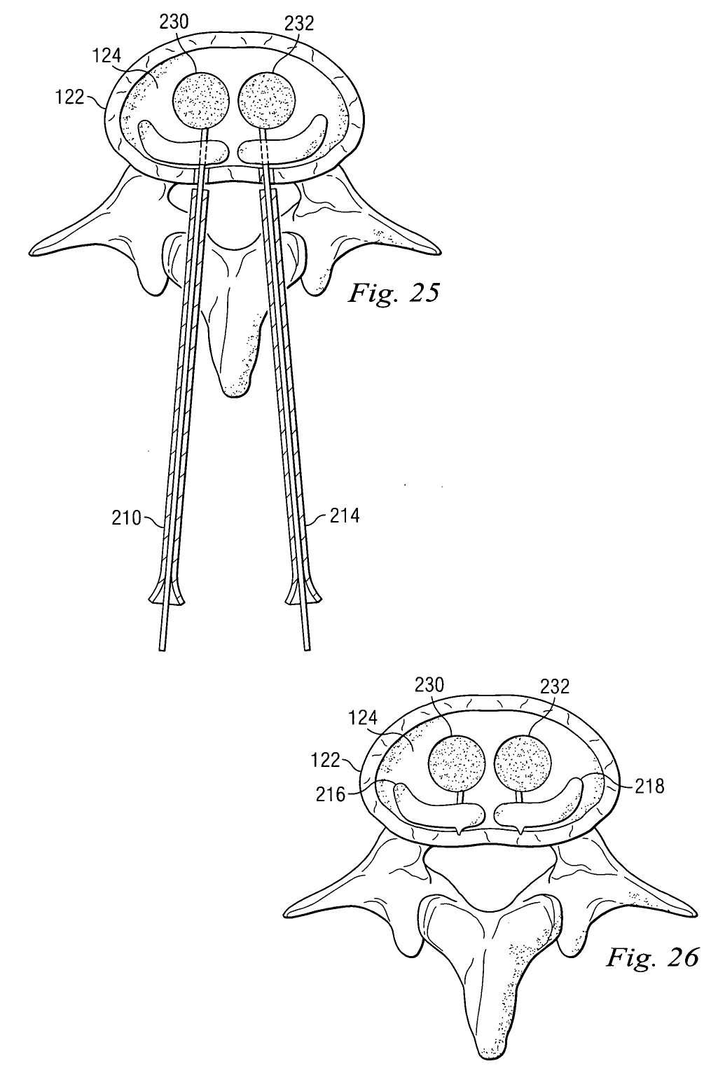

Referring now to FIGS. 25-26, in this embodiment, a substantially similar method of nucleus augmentation as the procedure described above for FIGS. 23-24 may be performed. In this embodiment, however, as described in FIGS. 19-20, spacing portions 230, 232 for creating the more centralized nucleus spaces may be detached to remain in the nucleus tissue as implants. The space creation and filling procedures of this embodiment may be controlled with a process and system similar to that described above for FIGS. 1 and 11. Although not shown, sensors similar to those described above for FIGS. 13-16 may be embedded in the instrumentation or disc space to monitor the space creation and filling.

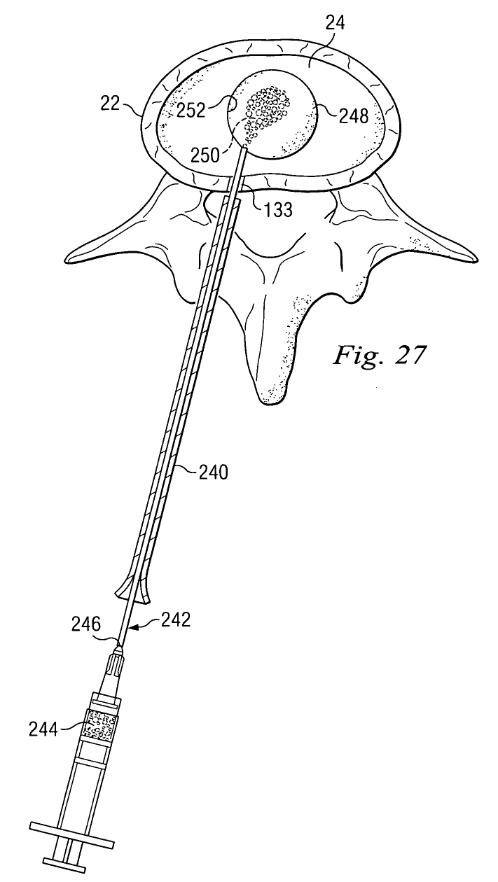

Referring now to FIG. 27, in this embodiment, a unilateral approach to augmenting a disc may be used. A cannula 240 may be inserted as described above. Through the cannula 240, a portion of a space creating device 242 may be inserted. The space creating device 242 has a delivery instrument 244, a catheter portion 246 and a spacing portion 248. In this embodiment, the spacing portion 242 may be expanded and filled with a biomaterial 250 to create a space 252. The spacing portion 242 may be detached and allowed to remain in the nucleus 24 as an implant. The space creation and filling procedures of this embodiment may be controlled with a process and system similar to that described above for FIGS. 1 and 11. Although not shown, sensors similar to those described above for FIGS. 13-16 may be embedded in the instrumentation or disc space to monitor the space creation and filling.

Although the instruments and implants described are suitable for intervertebral applications, it is understood that the same implants and instruments may be modified for use in other regions including an interspinous region or a bone cavity. Furthermore, the instruments and implants of this disclosure may be incorporated in certain aspects into an intervertebral prosthesis device such as a motion preserving artificial disc.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A system for controlling a nucleus pulposus augmentation procedure for an intervertebral disc, the system comprising:
a powered actuation device;
a control device for controlling an operating parameter of the actuation device;
a space creating instrument including
a spacing portion for forming a space within the nucleus pulposus of the intervertebral disc and
a delivery instrument for delivering a material to the spacing portion,
wherein the space creating instrument is activated by the powered actuation device to expand the spacing portion with the material to form the space within the nucleus pulposus of the intervertebral disc.

2. The system of claim 1 further comprising:
feedback sensors coupled to the space creating instrument and adapted to transmit a first data type to the control device.

3. The system of claim 1 wherein the first data type is pressure data.

4. The system of claim 1 wherein the first data type is material volume data.

5. The system of claim 1 wherein the space creating instrument further comprises
a catheter extending between the spacing portion and the delivery instrument and
a catheter sensor coupled to the catheter and adapted to transmit a second data type to the control device.

6. The system of claim 1 further comprising a spacing portion sensor coupled to the spacing portion and adapted to transmit a third data type to the control device.

7. The system of claim 1 further comprising an anatomic sensor adapted for implantation within the intervertebral disc and adapted to transmit a fourth data type to the control device.

8. The system of claim 1 further comprising an input menu adapted to receive at least one input parameter for operating the control device.

9. The system of claim 8 wherein the at least one input parameter includes a patient diagnosis.

10. The system of claim 8 wherein the at least one input parameter includes an injection media parameter.

11. The system of claim 8 wherein the at least one input parameter includes a biomaterial parameter.

12. The system of claim 8 wherein the at least one input parameter includes an automatic control parameter.

13. The system of claim 1 further comprising at least one expansion profile adapted to control the expansion of the spacing portion.

14. The system of claim 13 wherein the at least one expansion profile provides a linear expansion profile.

15. The system of claim 13 wherein the at least one expansion profile provides a curved expansion profile.

16. The system of claim 13 wherein the at least one expansion profile provides a step profile.

17. The system of claim 13 wherein the at least one expansion profile provides a sine wave profile.

18. The system of claim 13 wherein the at least one expansion profile provides a square wave profile.

19. The system of claim 1 wherein the powered actuation device is a motor.

20. The system of claim 1 wherein the operating parameter is a speed parameter.

21. The system of claim 1 wherein the spacing portion is a balloon.

22. The system of claim 1 wherein the delivery instrument is an injector.

23. A method for augmenting a nucleus pulposus of an intervertebral disc, the method comprising:
introducing a spacing device through an opening in an annulus fibrosis of the intervertebral disc;
connecting the spacing device to a material delivery instrument;
connecting the material delivery instrument to an actuator;
activating the actuator to dispense a material from the material delivery device into the spacing device; and
controlling the actuator with a control device in accordance with a preprogrammed profile.

24. The method of claim 23 wherein the material delivery instrument comprises at least one instrument sensor and the method further comprises sending a data type from the at least one instrument sensor to the control device.

25. The method of claim 23 wherein the material is curable in situ.

26. The method of claim 23 further comprising:
removing the material from the spacing device.

27. The method of claim 26 further comprising:
filling a space formed by the spacing device with a biocompatible material.

28. The method of claim 23 wherein the step of controlling the actuator comprises controlling the speed of the actuator.

29. The method of claim 23 further comprising measuring a pressure in the material delivery instrument and sending a pressure measurement to the control device.

30. The method of claim 23 further comprising measuring a volume change in the material delivery instrument and sending a volume measurement to the control device.

31. The method of claim 23 further comprising measuring a pressure in a catheter connecting the spacing device to the material delivery instrument and sending a pressure measurement to the control device.

32. The method of claim 23 further comprising measuring a pressure in the spacing device and sending a pressure measurement to the control device.

33. The method of claim 23 further comprising measuring a pressure in the intervertebral disc and sending a pressure measurement to the control device.

34. A method for augmenting a nucleus pulposus of an intervertebral disc, the method comprising:
forming a first opening in an annulus of the intervertebral disc;
forming a second opening in the annulus of the intervertebral disc;
providing a space creation instrument including an expandable spacing device;
introducing the spacing device through the first opening and into the nucleus pulposus;
introducing a material delivery instrument through the second opening and into the nucleus pulposus;
expanding the spacing device to create a space within the nucleus pulposus;
actuating the material delivery instrument to inject a biocompatible material into the space within the nucleus pulposus; and
controlling the injection of the biocompatible material with a first preprogrammed profile.

35. The method of claim 34 wherein the spacing device comprises an inflatable balloon.

36. The method of claim 34 further comprising controlling the expansion of the spacing device with a second preprogrammed profile.

37. The method of claim 34 further comprising controlling the injection of the biocompatible material with a user input received from an input menu.

38. The method of claim 34 further comprising controlling the injection of the biocompatible material with data received from a sensor located in the material delivery instrument.

39. The method of claim 34 further comprising controlling the injection of the biocompatible material with data received from a sensor located in the spacing device.

40. The method of claim 34 further comprising controlling the injection of the biocompatible material with data received from a sensor located in the intervertebral disc.
